(19) United States
(12) Patent Application Publication
(10) Pub. No.: US 2023/0395258 A1
(43) Pub. Date: Dec. 7, 2023

(54) SYSTEMS AND METHODS FOR PREDICTING TRANSPLANT REJECTION

(71) Applicant: CareDx, Inc., Brisbane, CA (US)

(72) Inventors: Kunbin QU, Los Altos, CA (US); Alexandre LOUPY, Paris (FR); Olivier AUBERT, Paris (FR)

(73) Assignee: CareDx, Inc., Brisbane, CA (US)

(21) Appl. No.: 18/328,651

(22) Filed: Jun. 2, 2023

Related U.S. Application Data
(60) Provisional application No. 63/348,971, filed on Jun. 3, 2022.
(60) Provisional application No. 63/358,484, filed on Jul. 5, 2022.

(51) Int. Cl. G16H 50/20; G16H 50/30; G16H 50/70

(52) U.S. Cl. CPC G16H 50/20; G16H 50/30; G16H 50/70; G16H 10/60

(57) ABSTRACT

Disclosed herein are computer-implemented methods and systems for predicting transplant rejection. The methods and system may calculate a predicted probability of whether or an extent to which transplant rejection in a transplant recipient will occur may be calculated based on a score. The score may be calculated based on transplant recipient data and one or more parameter weights. The transplant recipient data may comprise transplant donor-derived cell-free DNA (dd-cfDNA) and one or more other types of parameters, such as one or more clinical parameters, one or more functional parameters, one or more immunological parameters, one or more transplant recipient characteristics, or one or more transplant characteristics. In some embodiments, the parameters used for calculating the predicted probability does not comprise a histological parameter.

### CROSS-REFERENCE TO RELATED APPLICATIONS

This application claims the benefit of U.S. Provisional Application No. 63/348,971, filed on Jun. 3, 2022, and U.S. Provisional Application No. 63/358,484, filed on Jul. 5, 2022, both of which are hereby incorporated by reference in their entirety.

### FIELD OF THE DISCLOSURE

This disclosure relates generally to systems and methods for determining the status of an allograft, including predicting transplant rejection.

### BACKGROUND OF THE DISCLOSURE

Transplantation of cells, tissues, partial or whole organs are life-saving medical procedures in cases where an individual experiences acute organ failure or suffers from some malignancy. Many organs including, but not limited to, heart, kidney, liver, lung, and pancreas can be successfully transplanted, and one of the most common types of organ transplantations performed nowadays is kidney transplantation.

Upon transplantation of non-self (allogeneic) cells, tissues, or organs (allograft) into a recipient, the transplant recipient's immune system recognizes the allograft to be foreign to the body and activates various mechanisms to reject the allograft. Thus, it is necessary to medically suppress such an immune response to minimize the risk of transplant rejection. After transplantation, the status of the transplant may be monitored by a variety of clinical laboratory diagnostics tests, which may include invasive, histopathologic assessment of transplant biopsy tissue. A histopathological assessment (e.g., a biopsy), however, only provides information about the current rejection status of an allograft and lacks the ability to provide a prognostic and/or predictive risk evaluation with respect to future allograft dysfunction and/or allograft rejection.

Physicians or medical experts may have their own standards for weighting the contributions of measured information to the overall assessment of the status of an allograft. The measured information may be collected from various clinical laboratory diagnostics tests, demographic information, and other parameters. An objective and consistent quantification of the risk of transplant rejection, optionally in comparison to a reference set of allograft statuses, would be useful to guide treatment options, such as immunosuppressive therapies and daily clinical care.

What is needed are systems and methods for predicting transplant rejection based on objective, comprehensive, consistent, and comparative assessments that will ultimately improve diagnostic accuracy and reliability, as provided by the present disclosure.

### BRIEF SUMMARY OF THE DISCLOSURE

A computer-implemented method for determining a risk of transplant rejection using a machine-learning system is disclosed. The method comprises: receiving, via a computer or an input function, transplant recipient data of a transplant recipient comprising a set of parameters, the set of parameters comprising donor-derived cell-free DNA (dd-cfDNA); receiving one or more parameter weights; calculating a score based on the transplant recipient data and the one or more parameter weights; and calculating a predicted probability of whether or an extent to which transplant rejection in the transplant recipient will occur based on the score. Additionally or alternatively, in some embodiments, the set of parameters of the transplant recipient data further comprises one or more of: one or more clinical parameters comprising time post-transplantation to evaluation; one or more functional parameters comprising estimated glomerular filtration rate (eGFR), creatinine, or proteinuria; one or more immunological parameters comprising donor-specific antibody mean fluorescence intensity or number of anti-human leukocyte antigens (HLA) mismatches; one or more transplant recipient characteristics comprising transplant recipient age or donor organ infection information; or one or more transplant characteristics comprising prior transplant information or previous rejection information. Additionally or alternatively, in some embodiments, the set of parameters does not comprise a histological parameter. Additionally or alternatively, in some embodiments, the computer-implemented method, further comprises: generating a projection of the transplant recipient data in a reference set, wherein the reference set comprises one or more other transplant recipients having one or more common characteristics. Additionally or alternatively, in some embodiments, calculating a score comprises: for each parameter weight, multiplying the parameter weight by a corresponding parameter of the transplant recipient data; and calculating the score from a summation of the multiplications. Additionally or alternatively, in some embodiments, calculating a predicted probability comprises: determining an intercept of a multivariable logistic regression model; and calculating the predicted probability from the score and the intercept. Additionally or alternatively, in some embodiments, the one or more parameters weights are received from a machine-learning model trained to: acquire a cohort dataset comprising a first set of model parameters and cohort transplant rejection information of transplant recipients in a cohort, wherein the first set of model parameters comprises dd-cfDNA; analyze the first set of model parameters for associations between the cohort dataset and the corresponding cohort transplant rejection information, wherein the first set of model parameters are analyzed individually; select a second set of model parameters from the first set of model parameters, wherein the second set of model parameters meets one or more first criteria; select a third set of model parameters from the second set of model parameters, wherein the third set of model parameters comprises independent variables associated with transplant rejection and meets one or more second criteria; and generate the one or more parameter weights corresponding to the third set of model parameters of the cohort dataset. Additionally or alternatively, in some embodiments, the first set of model parameters further comprises one or more of: one or more clinical parameters comprising kidney graft dysfunction, time since last transplant rejection, or time from transplant to evaluation; one or more functional parameters comprising estimated glomerular filtration rate (eGFR) or proteinuria; one or more immunological parameters comprising donor-specific antibody mean fluorescence intensity or number of anti-human leukocyte antigens (HLA) mismatches; one or more recipient and donor characteristics comprising recipient age, recipient gender, or donor organ infection information; or one or more transplant characteristics comprising donor age, donor gender, donor type, prior transplant information, cold ischemia time, or dual transplant kidney information. Additionally or alternatively, in some embodiments, the second set of model parameters comprises dd-cfDNA, allograft dysfunction, recent transplant rejection information, time post-transplantation to evaluation, estimated glomerular filtration rate (eGFR), proteinuria, donor-specific antibody mean fluorescence intensity, recipient age, recipient gender, donor age, donor gender, donor type, prior transplant information, cold ischemia time, dual transplant information, and number of anti-human leukocyte antigens (HLA) mismatches. Additionally or alternatively, in some embodiments, wherein the third set of model parameters comprises dd-cfDNA, estimated glomerular filtration rate (eGFR), graft dysfunction, recent transplant rejection information, and donor-specific antibody mean fluorescence intensity. Additionally or alternatively, in some embodiments, the machine-learning model trained to analyze the first set of model parameters for associations comprises the machine-learning model trained to analyze whether or an extent to which a parameter of the first set of model parameters discriminates between a presence or an absence of transplant rejection in the cohort transplant rejection information. Additionally or alternatively, in some embodiments, the one or more first criteria or the one or more second criteria comprise a model parameter having a confidence interval greater than or equal to a confidence interval threshold or a p-value less than a p-value threshold. Additionally or alternatively, in some embodiments, the confidence interval threshold is 95% and the p-value threshold is 0.2. Additionally or alternatively, in some embodiments, the one or more first criteria comprise a number of model parameters in the second set of model parameters being less than a threshold number. Additionally or alternatively, in some embodiments, the machine-learning model trained to analyze the first set of model parameters for associations comprises the machine-learning model trained to reduce a dimensionality of the cohort dataset based on a presence or an absence of transplant rejection in the cohort transplant rejection information. Additionally or alternatively, in some embodiments, the machine-learning model trained to analyze the first set of model parameters for associations comprises the machine-learning model trained to determine associations between the dd-cfDNA and one or more of: cause of end stage renal disease or type of transplant rejection. Additionally or alternatively, in some embodiments, the machine-learning model trained to analyze the first set of model parameters comprises the machine-learning model trained to reduce a dimensionality of the cohort dataset based on a type of transplant rejection. Additionally or alternatively, in some embodiments, the machine-learning model trained to select the third set of model parameters comprises the machine-learning model trained to: perform backward selection by individually analyzing whether or an extent to which a parameter of the second set of model parameters discriminates between a presence or an absence of transplant rejection in the cohort transplant rejection information; and compare the individual analyses to select the third set of model parameters. Additionally or alternatively, in some embodiments, the one or more second criteria comprise a number of model parameters in the third set of model parameters being less than a threshold number.

A system for classifying a status of a transplant is disclosed. The system may comprise: a scoring unit that: receives transplant recipient data of a transplant recipient comprising a set of parameters, the set of parameters comprising donor-derived cell-free DNA (dd-cfDNA); receives one or more parameter weights; calculates a score based on the transplant recipient data and the one or more parameter weights; and calculates a predicted probability of whether or an extent to which transplant rejection in the transplant recipient will occur based on the score. The system may include one or more data processors and a non-transitory computer readable storage medium containing instructions which, when executed on the one or more data processors, cause the one or more data processors to perform part or all of one or more methods disclosed herein. In some embodiments, the system includes a non-transitory computer readable storage medium containing instructions which, when executed on the one or more data processors, cause the one or more data processors to perform part or all of one or more methods and/or part or all of one or more processes disclosed herein.

A computer-program product is disclosed. The computer-program product may be tangibly embodied in a non-transitory machine-readable storage medium and that includes instructions configured to cause one or more data processors to perform part or all of one or more methods disclosed herein.

### BRIEF DESCRIPTION OF THE DRAWINGS

FIG. 1 illustrates an example system for predicting transplant rejection, according to embodiments of the disclosure.

FIG. 2 illustrates a flowchart of an example method for calculating a predicted probability of transplant rejection in a transplant recipient and/or generating a projection of the transplant recipient data in a reference set, according to embodiments of the disclosure.

FIG. 3A illustrates an example user interface displaying text boxes and input boxes, according to embodiments of the disclosure.

FIG. 3B illustrates an example user interface displaying example predicted probability (panel A, left) and projection (panel B, right), according to embodiments of the disclosure.

FIGS. 3C and 3D illustrate example user interfaces for displaying predicted probabilities panel A, left) and projections (panel B, right) for additional example transplant recipients, according to embodiments of the disclosure.

FIG. 4A illustrates an example system for generating parameter weights, according to embodiments of the disclosure.

FIG. 4B illustrates a flow chart of an example method performed by a machine-learning model, according to embodiments of the disclosure.

FIG. 5 illustrates a table of example data for a second set of parameters of a cohort dataset selected from a first set of parameters, according to embodiments of the disclosure.

FIG. 6 illustrates a table of example data for a third set of parameters of a cohort dataset selected from a second set of parameters, according to embodiments of the disclosure.

FIG. 7 illustrates an example device that implements the above disclosed system and methods, according to embodiments of the disclosure.

### DETAILED DESCRIPTION

The present disclosure is based, at least in part, on the development of computer-implemented methods and systems for predicting transplant rejection.

Predicting a transplant recipient's risk of allograft rejection based on objective, comprehensive, consistent, and comparative assessments allows the classification and treatment of transplant recipients in accordance to their overall risk of allograft rejection, which, in turn, will improve daily clinical care, avoid unnecessary, invasive procedures and prolong allograft survival.

The predicted probability of whether or an extent to which transplant rejection in a transplant recipient will occur may be calculated based on a score. The score may be calculated based on transplant recipient data and one or more parameter weights. The transplant recipient data may comprise transplant donor-derived cell-free DNA (dd-cfDNA) and one or more other types of parameters, such as one or more clinical parameters, one or more functional parameters, one or more immunological parameters, one or more transplant recipient characteristics, or one or more transplant characteristics. In some embodiments, the parameters used for calculating the predicted probability does not comprise a histological parameter.

The predicted probability may be used by a physician or medical expert. The physician or medical expert may input the transplant recipient data into an interface. A scoring unit may receive the transplant recipient data and calculate a score based on the transplant recipient data and one or more parameter weights. The scoring unit may receive the parameter weight(s) by a trained machine-learning model. The scoring unit may also calculate the predicted probability. The predicted probability may be a binary indication (e.g., yes or no) or a quantitative value (e.g., 80%) indicative of an extent to which transplant rejection in the transplant recipient will occur. The predicted probability may be provided on a graphical user interface displayed to the physician or medical expert. The predicted probability may be an objective measure used complementary to, or in the alternative of, an assessment by the physician or medical expert.

The predicted probability may be provided by way of a medical analysis tool that is readily accessible to a physician or medical expert. The medical analysis tool may display a predicted probability, a projection, or both on a user interface. The predicted probability may be an accurate and quantifiable measure of the status of an allograft.

By quantifying the prediction, the predicted probability and projection may become objective and more consistent. The predicted probability can be used to accurately compare the status of an allograft at one point in time to another point in time. Additionally or alternatively, the quantification may be used as a guide for deciding treatment options and related timing. To provide consistency, reliability, and granularity, embodiments of the disclosure may include computer-implemented tools and methods for assessing the measurements from a transplant recipient. A systematic assessment may help to better characterize a transplant recipient's response to therapy and help inform subsequent management of the transplant recipient. The results of the machine-learning model and medical analysis tool may be more reproducible such that variations between transplant recipients or different measurement times for a given transplant recipient may be reduced.

The one or more parameter weights may be generated by a machine-learning model trained to acquire a dataset comprising a set of parameters and transplant rejection information from transplant recipients in a cohort. The machine-learning model may analyze the parameters and perform one or more selection steps to select a subset of the parameters. The machine-learning model may then generate the parameter weights corresponding to the selected subset of parameters.

The machine-learning model may be trained to select parameters associated with the status (e.g., predicted likelihood of failure) of an allograft. The machine-learning model may receive a cohort dataset from a cohort, such as a derivation cohort. The cohort dataset may comprise parameters that may or may not be associated with allograft failure. In some embodiments, the parameters may have different degrees of association. The machine-learning model may select those parameters that have the highest degree of association to transplant rejection. In some embodiments, the machine-learning model may generate the parameter weights in accordance with degrees of association. The parameters that are most associated with allograft failure may be weighted more.

The following description is presented to enable a person of ordinary skill in the art to make and use various embodiments. Descriptions of specific devices, techniques, and applications are provided only as examples. These examples are being provided solely to add context and aid in the understanding of the described examples. It will thus be apparent to a person of ordinary skill in the art that the described examples may be practiced without some or all of the specific details. Other applications are possible, such that the following examples should not be taken as limiting. Various modifications in the examples described herein will be readily apparent to those of ordinary skill in the art, and the general principles defined herein may be applied to other examples and applications without departing from the spirit and scope of the various embodiments. The various embodiments are not intended to be limited to the examples described herein and shown, but are to be accorded the scope consistent with the claims.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to a person of ordinary skill in the art, that one or more aspects and/or features described or referenced herein may be practiced without some or all of these specific details. In other instances, well-known process steps and/or structures have not been described in detail in order to not obscure some of the aspects and/or features described or referenced herein.

In the following description of examples, reference is made to the accompanying drawings which form a part hereof, and in which, by way of illustration, specific examples are shown that can be practiced. It is to be understood that other examples can be used and structural changes can be made without departing from the scope of the disclosed examples.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combination of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

### Example System for Predicting Transplant Rejection

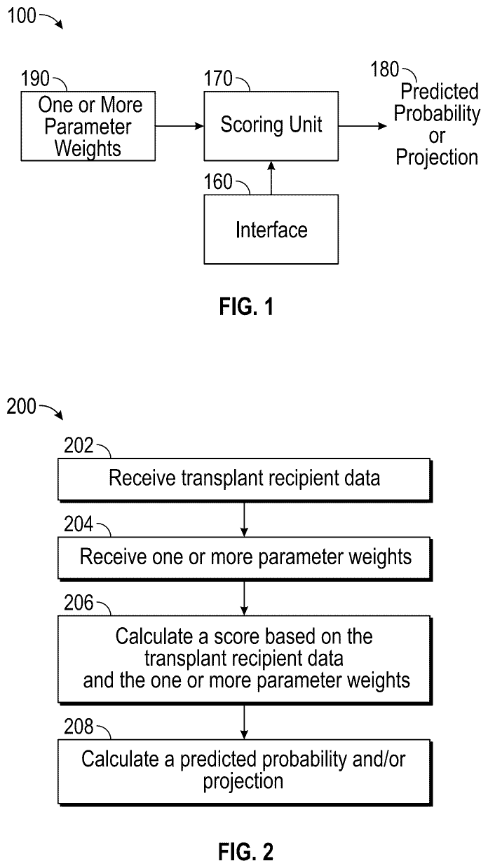

FIG. 1 illustrates an example system 100 for predicting transplant rejection, according to embodiments of the disclosure. System 100 may include an interface 160 and a scoring unit 170. Examples of the disclosure may include some or all of the components shown in the figure, or other components not shown in the figure. The system 100 may be, for example, a medical analysis tool. A physician or medical expert may use the medical analysis tool to help monitor and/or classify the status of an allograft in a transplant recipient, as well as monitor and/or suggest an adjustment to an immunosuppressive therapy administered, or to be administered, to a transplant recipient. Monitoring the status of an allograft involves analyzing various aspects that provide useful information about the physiological state or health status of the allograft. The methods of the present disclosure may be used to predict the probability of transplant rejection based on the measurement information indicative of the status of the allograft. The predicted probability may be a quantitative percentage reflecting the probability that transplant rejection will occur in the transplant recipient.

The interface 160 may receive user input (e.g., input from a physician or medical expert) of transplant recipient data. Example transplant recipient data may comprise information from laboratory tests relating to transplant donor-derived cell-free DNA (dd-cfDNA); information, at one or more time points regarding: creatinine levels in plasma, serum, and/or urine, proteinuria, estimated glomerular filtration rate, time post-transplantation until evaluation, transplant recipient characteristics such as age and gender, information regarding prior transplantations, information regarding prior transplant rejection events, etc. The interface 160 is discussed in more detail below.

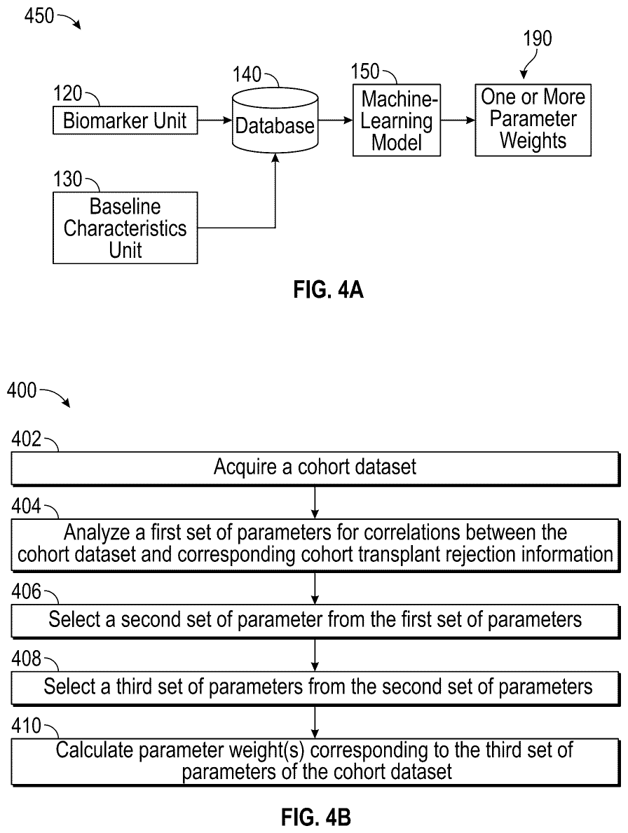

The scoring unit 170 may be a tool for assessing the transplant recipient data (e.g., information from laboratory tests obtained from a blood and/or urine sample of the transplant recipient or information from a biopsy). The scoring unit 170 may receive one or more parameter weights 190 (e.g., from a machine-learning model 150, shown in FIG. 4A) and the transplant recipient data from the interface 160. The scoring unit 170 may calculate the status of an allograft of the transplant recipient. The status of the allograft may include a predicted probability or a projection. The scoring unit 170 may output the predicted probability and/or projection 180.

FIG. 2 illustrates a flowchart of an example method for calculating a predicted probability of transplant rejection in a transplant recipient and/or generating a projection of the transplant recipient data in a reference set, according to embodiments of the disclosure. Method 200 may comprise step 202, where the system 100 may receive transplant recipient data. The transplant recipient data may be received by, e.g., interface 160. Transplant recipient data may be data associated with the transplant recipient. The transplant recipient data may comprise measurement information from the transplant recipient. Example measurement information may include, but is not limited to, information, at one or more time points, regarding: creatinine levels in plasma, serum, and/or urine, proteinuria, urine albumin, urine microalbumin, urine protein, estimated glomerular filtration rate (eGFR), urine albumin-creatinine-ratio, blood urea nitrogen, serum sodium, serum potassium, serum chloride, serum bicarbonate, serum calcium, serum albumin, complete blood count panel, liver function panel, lipid profile panel, a coagulation panel, magnesium, phosphorus, brain natriuretic peptide, hemoglobin, uric acid, endostatin, number of HLA mismatches, mean fluorescent intensity (MFI) of donor-specific antibodies (DSA), and transplant recipient characteristics such as the transplant recipient's age, the time from the transplant to the evaluation, whether the transplant recipient has had a previous transplant, information regarding systemic infection, such as information regarding viral infections, e.g., BK virus infection. Optionally, measurement information may also include information regarding whether the transplant recipient had previously experienced a transplant rejection event. In some embodiments, the evaluation time may be the duration (e.g., number of years) between when measurement information was extracted and the transplantation occurred.

In step 204, the system may receive one or more parameters weights. The one or more parameter weights may be received from a machine-learning model, for example. As discussed in more detail below, the machine-learning model may be trained to generate the parameter weights based on various input parameters, such as a transplant recipient cohort dataset and corresponding cohort transplant rejection information. In various embodiments, the machine learning model or algorithm to analyze various input parameters may include, but not be limited to, logistic regression; regularized classification models, such as Ridge, lasso and elastic net; nearest shrunken centroid; gradient boosted machines; random forests; support vector machines; k nearest neighbors; neural networks, and so forth.

In step 206, the system may use the scoring unit 170 to calculate a score based on the transplant recipient data and the parameter weight(s). The transplant recipient data may comprise a set of parameters. The set of parameters may comprise at least donor-derived cell-free DNA (dd-cfDNA). In some embodiments, the set of parameters may comprise a parameter related to information regarding whether the transplant recipient had previously experienced a transplant rejection event. The parameter weights may correspond to the set of parameters. In some embodiments, each parameter may have a corresponding parameter weight. In some embodiments, each parameter weight may indicate a degree of association of the parameter to transplant rejection. For example, a higher parameter weight may indicate a higher association of the parameter to transplant rejection. The score may be calculated based on the transplant recipient data and the parameter weights. In some embodiments, the score may be calculated by summing together a plurality of multiplications. Each multiplication may be for each parameter weight, comprising multiplying the parameter weight by a corresponding parameter of the transplant recipient data. The generation of the weights is discussed in more detail below.

In step 208, the system may determine the status, e.g., the health status, of the allograft. The health status may be a status indicating allograft tolerance or allograft rejection, or the likelihood thereof. In some embodiments, the determination of the status of the allograft may comprise calculating a predicted transplant rejection probability and/or generating a projection. The predicted probability may be a numerical value (e.g., a percentage) indicative of whether or an extent to which transplant rejection in the transplant recipient will occur based on the score. In some embodiments, calculating the predicted probability comprises calculating a probability from the score. In some embodiments, calculating the predicted probability comprises determining an intercept of a multivariable logistic regression model, and calculating the predicted probability from the score and the intercept. The predicted probability may be represented by a numerical value, a level or category indicative of a numerical value, a binary indicator, or the like. In some embodiments, the predicted probability and/or projection may be indicative of the current risk of transplant rejection in the transplant recipient.

The projection may be a visual aid used for illustrating the transplant recipient's predicted probability in a reference set. The reference set may comprise information from one or more other transplant recipients having one or more common characteristics, such as the post-transplantation to the evaluation time.

Embodiments of the disclosure may include repeating one or more steps of method 200 and/or method 400 (discussed below). Although the descriptions and figures show particular steps of the method occurring in a particular order, the steps of the method may occur in other orders not described or shown. Additionally or alternatively, embodiments of the disclosure may include performing all, some, or none of the steps of method 200 and/or method 400, where appropriate. Furthermore, although certain components, devices, or systems are described as carrying out the steps of method 200 and/or method 400, any suitable combination of components, devices, or systems (including ones not explicitly disclosed) may be used to carry out the steps.

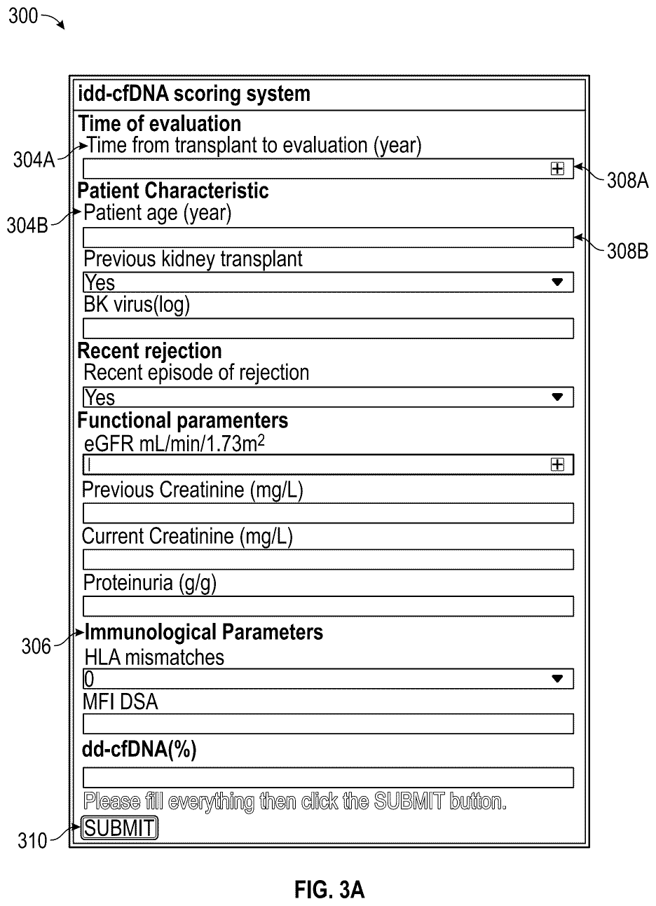

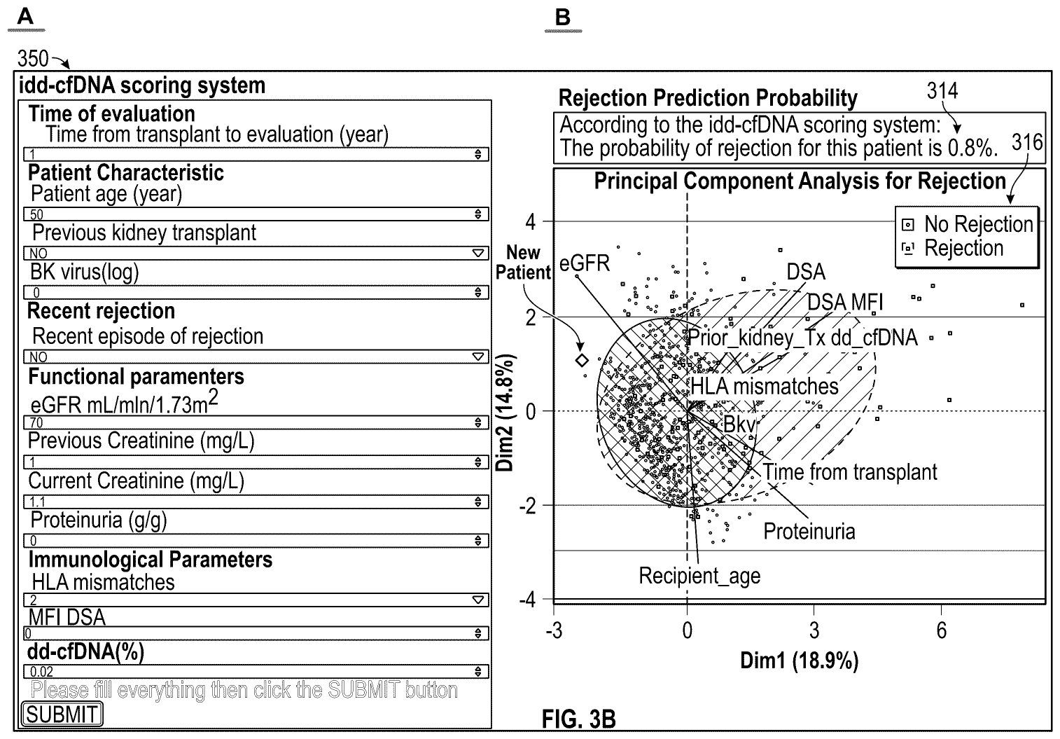

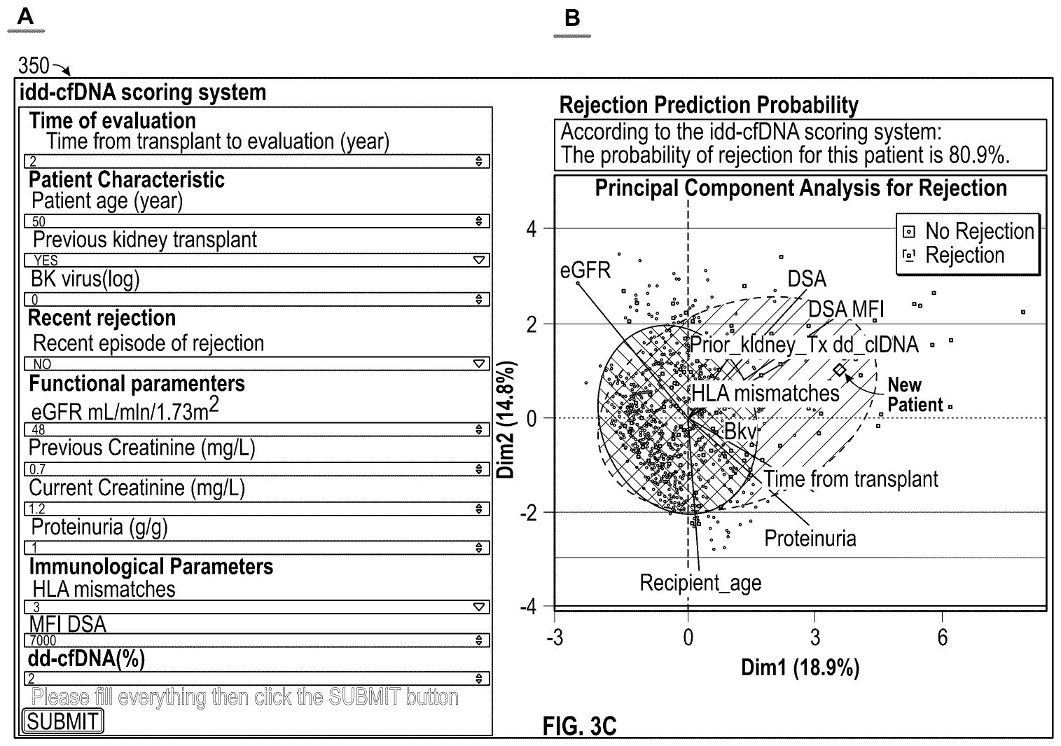

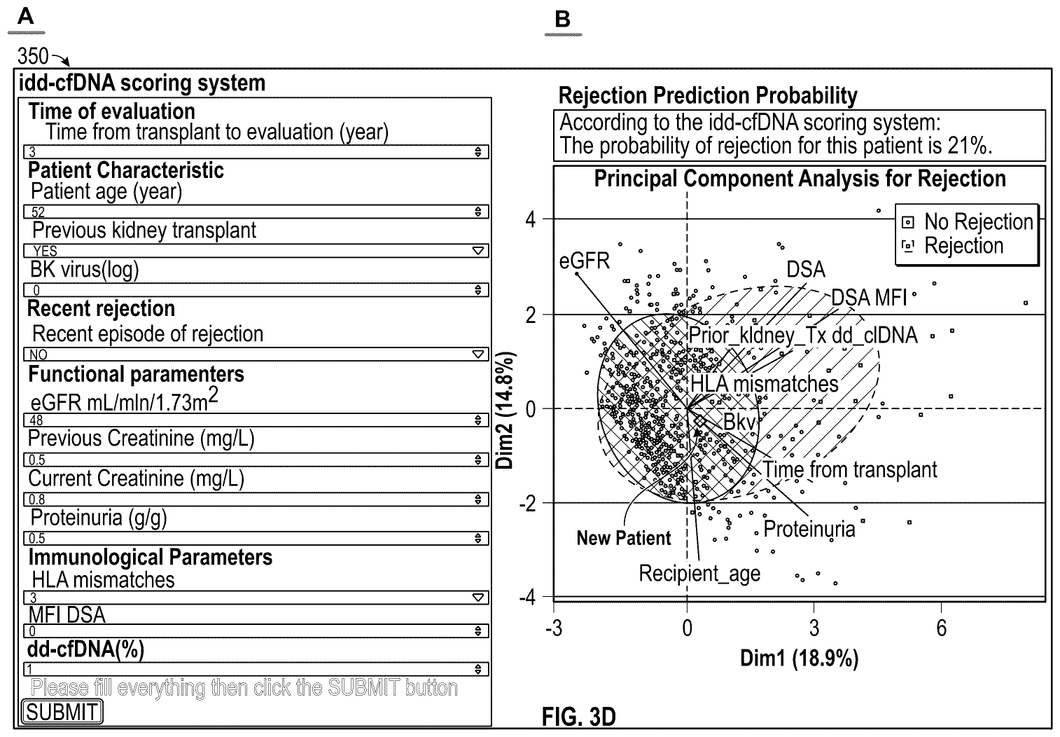

In some embodiments, the system may output the predicted probability and/or projection, such as displaying the predicted probability and/or projection on a user interface. The user interface may be included in a medical analysis tool that is readily accessible and immediately provides the status of an allograft to a physician or medical expert. FIG. 3A illustrates an example user interface displaying text boxes and input boxes, according to embodiments of the disclosure. The user interface 300 may be a user interface (UI) displayed on the display of a device (e.g., a mobile phone, a tablet, a laptop computer, etc.). In some embodiments, the user interface 300 may be accessed by having the user navigate to a website or application page.

The user interface 300 may include one or more text boxes 304, one or more input boxes 308, or both. The text of a given text box 304 may be associated with the type of information to be input by a user in a corresponding input box 308. For example, text box 304A may display “Time from transplant to evaluation (year).” The system may receive input from the user (e.g., a physician, nurse, medical assistant, medical expert, etc.) in the corresponding input box 308A. As another example, text box 304B may display “Patient age (year),” and corresponding input box 308B may be used by the user for inputting corresponding transplant recipient data. The transplant recipient data may comprise a (third) set of parameters used for calculating the predicted probability of whether or an extent to which transplant rejection in the transplant recipient will occur. As shown in the figure, example transplant recipient data may comprise one or more of the following parameters: time from transplantation to evaluation (“Time from transplant to evaluation (year)”), the transplant recipient's age (“Patient age (year)”), whether or not the transplant recipient has had a previous transplant, for example a previous kidney transplant (“Previously kidney transplant”), information regarding systemic infection, for example infection with a BK virus, (“BK virus (log)”), whether or not the transplant recipient had a recent episode of rejection (“Recent episode of rejection”), eGFR (“eGFR mL/min/1.73 m²”), information, one or more timepoints, regarding serum, plasma, and/or urine creatinine values (“Previous creatinine (mg/L)”), current creatinine values (“Current creatinine (mg/L)”), proteinuria values (“Proteinuria (g/g)”), number of HLA mismatches (“HLA mismatches”), MFI DSA (“MFI DSA”), or percentage of dd-cfDNA (“dd-cfDNA (%)”).

The user interface 300 may comprise one or more text boxes 306 that provide additional information, such as time of evaluation, transplant recipient characteristics, transplant recipient data, transplant characteristics, recent rejection, functional parameters, and/or immunological parameters, etc.) for the text boxes 304.

Additionally or alternatively, the user interface may comprise one or more graphical user interface buttons 310. The user interface button(s) 310 and the input boxes 308 may be interactive, enabling the user to input data and parameters, such as time of evaluation, transplant recipient characteristics, transplant recipient data, transplant characteristics, recent rejection, functional parameters, and/or immunological parameters, etc. After the user inputs information into the input boxes 308, the user may actuate the system to calculate the predicted probability and/or generate the projection 180, for example, by clicking on the “Submit” graphical user interface button 310. The direction and magnitude of the projected data and/or parameters, for example, in a bi-dimensional plot, as shown in FIGS. 3B-D, may provide further insight to elucidate or interpret a transplant recipient's rejection mechanism and guide corresponding patient management and treatment strategies.

FIG. 3B illustrates an example user interface displaying example predicted probability and projection, according to embodiments of the disclosure. The user interface 350 may comprise text boxes and input boxes similar to the user interface 300 (of FIG. 3A). In the example shown in FIG. 3B, the transplant recipient data has the parameters shown in Table 1.

TABLE 1
Example Parameters and Transplant Recipient Data

| Set of Parameters | Transplant Recipient Data | |
|---|---|---|
| time from transplant to evaluation | 1 | year |
| transplant recipient (patient) age | 50 | years old |
| previous kidney transplant information | No | |
| BK virus information | 0 | |
| recent episode of rejection | No | |
| eGFR | 70 mL/min/1.73 m² | |
| previous creatinine | 1 | mg/L |
| current creatinine | 1.1 | mg/L |
| proteinuria | 0 | g/g |

| | |
|---|---|
| HLA mismatches | 2 |
| MFI DSA | 0 |
| dd-cfDNA | 0.02% |

The user interface 350 may further comprise one or more text boxes and/or one or more graphical representations. Text box 314 may provide an output of the predicted probability, and graphical representation 316 may provide a visual aid of the projection. For example, as shown in the figure, from the example parameters and transplant recipient data, the probability of rejection for the transplant patient is 0.8%.

FIGS. 3C and 3D illustrate example user interfaces for displaying predicted probabilities and projections for additional example transplant recipients, according to embodiments of the disclosure. In FIG. 3C, the example transplant recipient is a 50 year old female who received a transplant two years ago. The transplant recipient did not have a recent rejection or donor organ infection, for example, with a BK virus, but did have a previous kidney transplant. Her previous creatinine was 0.7 mg/L, but current creatinine is 1.2 mg/L, showing renal dysfunction. Her eGFR is 48 mL/min/1.73 m² and proteinuria is 1 g/g. The number of HLA mismatches is three, anti-HLA DSA MFI is 7000, and dd-cfDNA is 2%. The system calculated a predicted probability of 80.9% and may classify her as having a high risk of rejection.

In FIG. 3D, the example transplant recipient is a 52 year old female who received her second kidney transplant, but has not had a recent rejection before. The time from transplant to evaluation was three years. The transplant recipient's previous and current creatinine values are 0.5 and 0.8 mg/L, respectively; eGFR is 48 mL/min/1.73 m², and proteinuria is 0.5 g/g. The number of HLA mismatches is three. The patient has no MFI DSA, and the dd-cfDNA is 1%. The calculated predicted probability for this transplant recipient is 21%, as shown in the figure.

As discussed above, the system 100 (e.g., a medical analysis tool) may generate one or more parameter weights. In some embodiments, the one or more parameter weights may be generated by a system 100. The parameter weights are used to calculate the predicted probability of transplant rejection or projection of a given transplant recipient.

FIG. 4A illustrates an example system for generating parameter weights, according to embodiments of the disclosure. System 450 may comprise a biomarker unit 120, a baseline characteristics unit 130, a database 140, and a machine-learning model 150.

The biomarker unit 120 can be configured to measure one or more characteristics of the samples from a transplant recipient. Example characteristics may include, but are not limited to, donor-derived cell-free DNA (dd-cfDNA), anti-human leukocyte antigen (HLA) donor specific antibodies (DSAs), and creatinine levels. dd-cfDNA may be expressed as a percent of the total cell-free DNA or as an absolute concentration. Anti-HLA DSAs may refer to the presence of donor-specific anti-HLA antibodies in the transplant recipient. Creatinine levels refer to the amount of chemical waste product filtered by the kidneys of a transplant recipient. In some embodiments, the blood samples and corresponding characteristics may be obtained at the same time as a biopsy.

In some embodiments, the characteristics of the samples from a transplant recipient may be determined following an experimental (laboratory) workflow involving extraction of cell-free DNA, comprising total cell-free DNA, namely, cell-free DNA from the transplant recipient and the transplant, from blood, plasma, serum and/or urine samples obtained from transplant recipients in a derivation cohort and a validation cohort (the cohorts are discussed in more detail below). In some embodiments, a level or amount of transplant donor-derived cfDNA (dd-cfDNA) may be determined involving targeted amplification and targeted high-throughput sequencing of selected polymorphic markers, e.g., selected single nucleotide polymorphisms (SNPs), as described in U.S. patent application Ser. No. 14/658,061, filed Mar. 13, 2015, and U.S. patent application Ser. No. 17/351,040, filed Jun. 17, 2021, respectively, both of which are hereby incorporated by reference in their entirety. Polymorphic markers represent loci where two or more alternative nucleic acid sequences or alleles occur, due to a change of one or more bases, one or more insertions, one or more repeats, one or more deletions, and variations thereof. Accordingly, in some embodiments, a level or amount of transplant donor-derived cfDNA (dd-cfDNA) may be determined involving targeted analysis of alternative polymorphic markers, such as short tandem repeats (STRS), restriction fragment length polymorphisms (RFLPs), variable number of tandem repeats (VNTR's), hypervariable regions, minisatellites, dinucleotide repeats, trinucleotide repeats, tetranucleotide repeats, simple sequence repeats, and insertion elements In some embodiments, dd-cfDNA may be quantified as a percentage of total cfDNA or as an absolute concentration. In some embodiments, the level or amount of dd-cfDNA and the characteristics of the samples from a transplant recipient may be determined following the receipt of experimental data, such as sequencing reads, or other data-related information, such as quality control-related data, results, genotype information, SNP mutation rate, and so forth, from a database or other non-experimental source.

Additionally or alternatively, the biomarker unit 120 may determine the mean fluorescence intensities (MFIs) of the DSAs. The MFI may indicate the donor-specific antibody strength. In some embodiments, beads can be categorized according to predetermined normalized MFIs, e.g., less than 500, 500-3000, 3000-6000, and greater than 6000. Single-antigen flow bead assay may be used to determine whether there are DSAs present against one or more antigens (e.g., HLA-A, HLA-B, HLA-Cw, HLA-DR, HLA-DQ, HLA-DP, etc.). Embodiments of the disclosure may include using HLA typing of the transplant recipients and donors to identify HLA antigens. The parameters for the predicted probability of transplant rejection may include one or more immunological variables, such as circulating anti-HLA DSAs.

In some embodiments, the biomarker unit 120 may measure or calculate one or more functional parameters. One non-limiting example functional parameter is estimated glomerular filtration rate (eGFR). The glomerular filtration rate (GFR) refers to the rate that a kidney filters blood and can be indicative of its renal function. Another non-limiting example functional parameter is proteinuria, which is the level of excess protein in the urine of a transplant recipient. Yet another example functional parameter is the time from transplantation to (risk) evaluation. In some embodiments, the time of risk evaluation may be the time when the biopsy or blood sample is extracted from the transplant recipient or measured.

The baseline characteristics unit 130 can be configured to receive, store, and/or determine one or more baseline characteristics specific to a given transplant recipient. Example baseline characteristics may include, but are not limited to, donor comorbidity, recipient comorbidity, recipient characteristics, e.g., age, gender, height, weight, cause of end stage renal disease including, but not limited to, glomerulopathy, polycystic kidney disease, diabetes, vascular disease, and other, previous transplant information (including information regarding previous transplantation), dual transplant information (including dual kidney transplant information), donor characteristics (e.g., age, gender, height, weight, deceased or not, expanded criteria donor, etc.), and transplant characteristics (e.g., cold ischemia time, HLA-A/B/DR mismatch, ABO incompatible transplantation, weight of transplant, etc.). The parameters for the predicted probability of transplant rejection may include one or more baseline characteristics.

The database 140 may be configured to store one or more datasets based on data from the biomarker unit 120 and/or baseline characteristics unit 130. For example, the database 140 may store a cohort dataset such as a derivation cohort dataset and a validation cohort dataset. The cohort dataset may include measurement information from a sample obtained from a transplant recipient who was the recipient of an allograft (e.g., organ transplant, tissue transplant, cell transplant, etc.) from a donor. A derivation cohort dataset may comprise data from transplant recipients having a first characteristic (e.g., received a transplant during a first time period), and a validation cohort dataset may comprise data from transplant recipients having a second characteristic (e.g., received a transplant during a second time period). In some embodiments, the one or more parameters (e.g., baseline characteristics, mean dd-cfDNA levels or concentrations, diagnoses, transplant rejection information, etc.) of the transplant recipients in the derivation cohort may be similar (e.g., mean values plus or minus a certain percent deviation) to the baseline characteristics of the transplant recipients in the validation cohort.

Transplanted organs may include, for example, heart, kidney, lung, liver, pancreas, cornea, organ system, a vascularized composite allograft transplant, intestinal transplant, or other solid organs, and combinations thereof. The transplant received by the recipient from the donor may also include other allografts such as, for example, a bone marrow transplant, pancreatic islet cells, stem cells, skin tissue, skin cells, or a xenotransplant.

In some embodiments, the allograft is a cellular allograft, e.g., a transplant comprising allogeneic cells that originate from a donor. These include, but are not limited to, cells taken directly from a donor for administration into a recipient, cells taken from a donor and genetically engineered before administration into a recipient, cells taken from a donor and cultured before administration into a recipient, cells taken from a donor and subjected to a manufacturing process before administration into a recipient, and any combination thereof. Cells may also be stored before administration into a recipient (“off-the-shelf” cells).

The recipient of the transplant may have received one or more of a variety of allogeneic cells. Allogeneic cells may include, but are not limited to, blood cells, stem cells, cardiomyocytes, neurons, lymphocytes, NK cells, NKT cells, T reg cells, macrophages, dendritic cells, and pancreatic islet cells. In some embodiments, the allogeneic cells are allogeneic blood cells. Allogeneic blood cells may include hematopoietic stem cells (HSCs), T cells, B cells, and CAR T cells, NK cells, NKT cells, or TILs. In some embodiments, the allogeneic cells are allogeneic T cells. In some embodiments, allogeneic cells are administered as bone marrow, cord blood, or purified allogeneic cells. In some embodiments, the allogeneic cells are bone marrow cells. In some embodiments, the allogeneic cells are cord blood cells. In some embodiments, the allogeneic cells are allogeneic CAR T cells, allogeneic universal CAR T cells (i.e., where the CAR binds to an antibody that binds a specific antigen), allogeneic split CAR T cells (i.e., where a dimerizing agent activates CAR T cell function), allogeneic activatable CAR T cells, allogeneic repressible CAR T cells, allogeneic multiphasic CAR T cells (i.e., where the CAR must bind multiple specific antigens and/or agents to induce T cell activation), allogeneic tumor infiltrating lymphocytes, allogeneic regulatory T cells, allogeneic genetically modified T cells, or allogeneic T cells with genetically modified or synthesized T cell receptors (TCRs), virus-specific T cells (e.g., EBV, HPV, BKV, CMV, etc.), antigen-specific T cells, neoantigen-specific T cells, or any cell isolated from a donor. In some embodiments, the allogeneic cells are derived from a donor. In some embodiments, the allogeneic cells are a cell allograft.

The database 140 may also store cohort transplant rejection information. The cohort transplant rejection information may comprise rejection outcomes of transplant recipients in a corresponding cohort. In some embodiments, the rejection outcome may be based on whether or not an allograft history of a transplant recipient showed a pattern of lesions that fulfilled Banff criteria for active AMR (A-AMR), chronic-active AMR (CA-AMR), acute TCMR, or chronic active TCMR (CA-TCMR). The cohort dataset acquired from the database 140 may comprise data corresponding to individual transplant recipients of the cohort and/or representative data (e.g., mean value, average value, median value, percentage, etc.) for a group of the transplant recipients of the cohort (including all or less than all of the transplant recipients in the cohort).

The machine-learning model 150 may be configured to receive a cohort dataset from the database 140, analyze one or more sets of parameters associated with the dataset, select one or more sets of parameters, and generate one or more parameter weights. The machine-learning model 150 may be trained to identify associations between the cohort dataset and the status of an allograft, and then generate the parameter weights based on the identified associations. The machine-learning model 150 may narrow down the set of parameters used in generating the weights by performing one or more selection steps.

FIG. 4B illustrates a flow chart of an example method performed by a machine-learning model, according to embodiments of the disclosure. Method 400 may comprise acquiring a cohort dataset from, e.g., a database 150, in step 402. The cohort dataset may comprise a first set of parameters and cohort rejection information of transplant recipients in a cohort. The cohort dataset may be data obtained by one or more units, such as biomarker unit 120 and baseline characteristics unit 130. The first set of parameters may comprise, but is not limited to, dd-cfDNA and one or more of: clinical parameters, functional parameters, immunological parameters, recipient characteristics, or transplant characteristics.

The clinical parameters may comprise allograft dysfunction, time since last transplant rejection, if any prior rejection occurred, or time from post-transplantation until evaluation. The allograft dysfunction, e.g., kidney allograft dysfunction, may refer to the increase of serum creatinine of more than 0.3 mg per liter or more than 50% from baseline serum creatinine. The baseline serum creatinine may be the lowest serum creatinine value for a given transplant recipient during, e.g., the month prior to evaluation, or the last known serum creatinine.

The functional parameters may comprise eGFR, creatinine, or proteinuria. The immunological parameters may comprise anti-human leukocyte antigens (HLA) donor-specific antibody mean fluorescence intensity or number of HLA mismatches. The recipient characteristics may comprise recipient age or recipient gender. The transplant characteristics may comprise transplant mass, donor age, donor gender, donor type (living related, deceased related, living non-related, deceased non-related), donor weight, donor height, prior transplant information, e.g., prior kidney transplant information, cold ischemia time, or dual transplant information, e.g., dual transplant kidney information.

In step 404, the machine-learning model 150 may analyze the first set of parameters for associations between the cohort dataset and the corresponding cohort transplant rejection information. For each parameter in the first set of parameters, the machine-learning model may determine whether there is an association with the corresponding cohort transplant rejection information. This step may comprise analyzing whether a parameter of the first set of parameters discriminates between the presence or absence of transplant rejection in the rejection information. In some embodiments, the first set of parameters may be analyzed individually from one another. That is, each parameter may be analyzed without accounting for the other parameters in the first set of parameters.

As a non-limiting example, the cohort may be a derivation cohort comprising 637 transplant recipients. The mean values from the cohort dataset are shown in Table 2. The machine-learning model 150 may analyze the diagnoses of the transplant recipients to determine that a particular parameter is not associated with transplant rejection. For example, of the 637 transplant recipients, 85 were diagnosed with active AMR, 23 with chronic AMR, seven with inactive AMR, 16 with acute TCMR, three with chronic active TCMR, seven with mixed rejection, 12 with borderline lesions, 14 with viral nephritis, 12 with glomerulonephritis without rejection, 12 with FSGS, 219 with IFTA, and 227 with no specific lesions. The machine-learning model may determine that there was little or no discrimination between the presence or absence of transplant rejection. As another example, the machine-learning model 150 may analyze the anti-HLA DSA MFI to determine that this parameter is associated with transplant rejection. For example, of the 637 transplant recipients, 383 transplant recipients had an anti-HLA DSA MFI less than 500, 194 transplant recipients had between 500-3000, 27 transplant recipients had between 3000-6000, and 33 transplant recipients had greater than 6000. The machine-learning model may determine there is an association between the anti-HLA DSA MFI parameter and the presence of transplant rejection in the cohort dataset.

dd-cfDNA levels or concentrations above a threshold or cut-off value may be associated with transplant rejection. In some embodiments, analyzing the first set of model parameters for associations comprises determining associations between dd-cfDNA levels or concentrations and one or more of: the cause of end stage renal disease or type of transplant rejection.

In some embodiments, the machine-learning model 150 may determine that there is a relatively low association between dd-cfDNA and one or more histological parameters. Embodiments of the disclosure include the set of parameters, used in calculating the score and predicted probability and/or generating the projection, as not comprising a histological parameter.

In step 406, the machine-learning model 150 may select a second set of parameters from the first set of parameters. The second set of parameters may be selected based on meeting one or more (first) criteria. The machine-learning model 150 may select one parameter at a time for evaluation. For example, the machine-learning model 150 may evaluate a first parameter to determine whether or not it meets the first criteria. The machine-learning 150 may then evaluate a second parameter and determine whether or not it meets the first criteria. In some embodiments, the analysis (step 404) and selection (step 406) of the first set of parameters may be performed together such that the results from the analysis may lead to the selection.

The selection of the parameters (including first, second, and/or third sets of parameters) may be such that an association exists between the selected parameter and transplant rejection. The level of association between a parameter and transplant rejection may be represented by one or more statistical probabilities, such as the odds ratio (OR), confidence interval (CI), or p-value. The odds ratio may quantify the strength of association between a parameter and transplant rejection. The confidence interval may represent that probability that a parameter is within a certain interval around the mean plus or minus the standard of deviation. For example, a confidence interval of 95% may mean that 95% of the data of a given parameter has a value that is around the mean value plus or minus the standard deviation.

The p-value may be a measure of how probable a difference between two sets of data is real. The p-value may represent the difference between the presence (first data set) and the absence (second data set) of transplant rejection. A smaller p-value may correspond to a larger difference. For example, a smaller p-value for a corresponding parameter means that the parameter has a greater amount of discrimination between the presence and absence of transplant rejection in the rejection information.

The one or more first criteria may comprise a parameter having a confidence interval greater than or equal to a confidence interval threshold, a p-value less than a p-value threshold, an odds ratio greater than an odds ratio threshold, or a combination thereof. A threshold or threshold value generally refers to any predetermined level or range of levels that is indicative of a parameter's association to the presence or absence of risk of transplant rejection in the transplant recipient. The threshold value can take a variety of forms. It can be single cut-off value, such as a median or mean.

The confidence interval threshold and p-value threshold may be pre-determined values, such as 95% and 0.2, respectively. In other words, those parameters that have a confidence greater than 95% and a p-value less than 0.2 may be selected to form the second set of parameters.

Embodiments of the disclosure may include other criteria used to select the second set of parameters, such as the number of parameters in the second set of parameters being less than a threshold number of parameters. The non-selected parameters may not be considered in subsequent steps, and thus, may not be included in the calculation of the predicted probability. In some embodiments, the number of parameters in the second set of parameters may be less than the number of parameters in the first set of parameters.

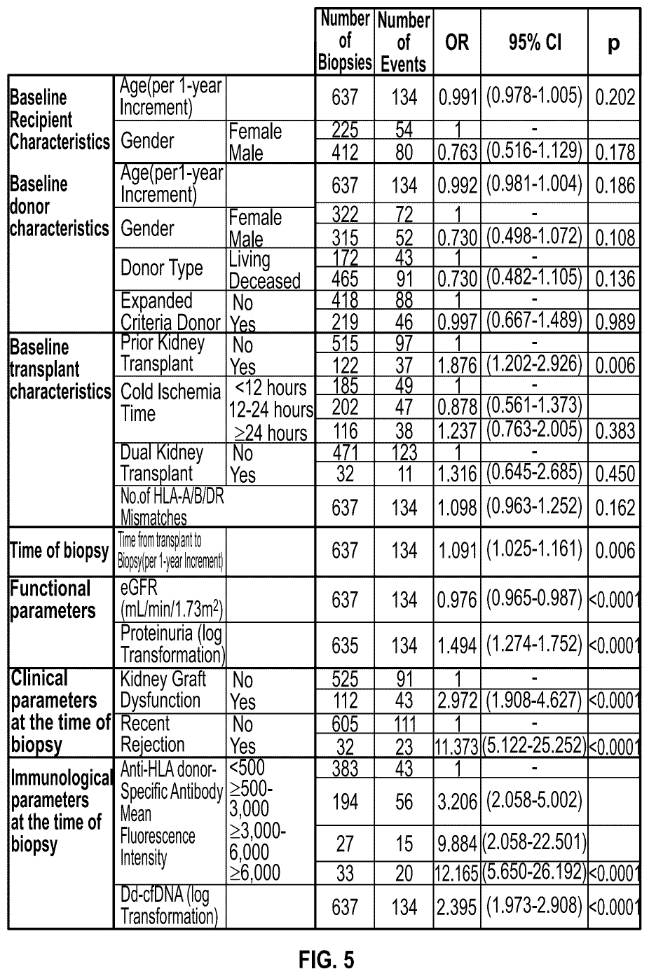

FIG. 5 illustrates a table of example data for a second set of parameters of a cohort dataset selected from a first set of parameters, according to embodiments of the disclosure. Example parameters in the second set of parameters may include, but are not limited to, dd-cfDNA levels or concentrations, allograft dysfunction, recent transplant rejection information, time from post-transplantation to evaluation, eGFR, proteinuria, anti-human leukocyte antigens donor-specific antibody mean fluorescence intensity, recipient age, recipient gender, donor age, donor gender, donor type, prior kidney transplant information, cold ischemia time, dual transplant kidney information, and number of HLA mismatches. Further parameters may include information from a biopsy.

Referring back to FIG. 4B, in step 408, the machine-learning model may select a third set of parameters from the second set of parameters. The third set of parameters may comprise independent variables associated with transplant rejection. In some embodiments, the third set of parameters may be selected based on meeting one or more second criteria. In some embodiments, the selection of the third set of parameters may comprise performing backward selection where a parameter of the second set of parameters may be individually analyzed to determine whether the parameter discriminates between the presence or absence of transplant rejection in the rejection information. The individual analysis may involve temporarily removing the parameter of interest to determine whether it has an impact on the level of association. In some embodiments, each parameter may be analyzed, and then the analyses may be compared to select the third set of parameters. For example, a first analysis may involve temporarily removing the eGFR parameter and determining that the level of association is lower with the remaining set of parameters. The machine-learning model 150 may select the eGFR parameter as part of the third set of parameters. A second analysis may involve temporarily removing the time from transplant to biopsy parameter and determining that the level of association did not change significantly, and as a result, the machine-learning model 150 may not select the time from transplant to biopsy parameter as part of the third set of parameters.

The one or more second criteria may comprise a parameter having a confidence interval greater than or equal to a confidence interval threshold, or a p-value less than a p-value threshold. In some embodiments, the confidence interval threshold and p-value threshold may be pre-determined values, such as 95% and 0.2, respectively. In other words, those parameters that have a confidence greater than 95% and a p-value less than 0.2 may be selected to form the second set of parameters. In some embodiments, the one or more second criteria may be the same as the one or more first criteria. In other embodiments, the one or more second criteria may have a p-value threshold that is less than the p-value threshold in the one or more first criteria.

Additionally or alternatively, other criteria may be used to select the third set of parameters. For example, the machine-learning model 150 may select the third set of parameters based on the number of parameters in the third set of parameters being less than a pre-determined number of parameters. The selected third set of parameters may be those parameters having the lowest p-value, for example, among the second set of parameters (e.g., N number of parameters having the lowest p-value). The non-selected parameters may not be considered in subsequent steps, and thus, may not be included in the calculation of the predicted probability. In some embodiments, the number of parameters in the third set of parameters may be less than the number of parameters in the second set of parameters.

The machine-learning model 150 may determine that the third set of parameters are highly associated with transplant rejection. For example, the machine-learning model 150 may select parameters that have the highest association with transplant rejection. These parameters may comprise, for example, certain baseline characteristics (e.g., transplant recipient age, previous transplant information, time from post-transplantation to evaluation), immunological variables (e.g., number of HLA mismatches, dd-cfDNA, anti-HLA DSA information), and information regarding systemic infection with viruses, bacteria, fungi, and parasites, which a transplant recipient might have contracted during the process of transplantation or following transplantation, such as BK virus information. Infectious agents that may cause a systemic infection and for whose presence or levels may be tested for may include, but are not limited to, viruses such as Cytomegalovirus, Epstein-Barr virus, Anelloviridae, and BK virus; bacteria such as Pseudomonas aeruginosa, Enterobacteriaceae, Nocardia, Streptococcus pneumonia, Staphyloccous aureus, and Legionella; fungi such as Candida, Aspergillus, Cryptococcus, Pneumocystis carinii; or parasites such as Toxoplasma gondii.

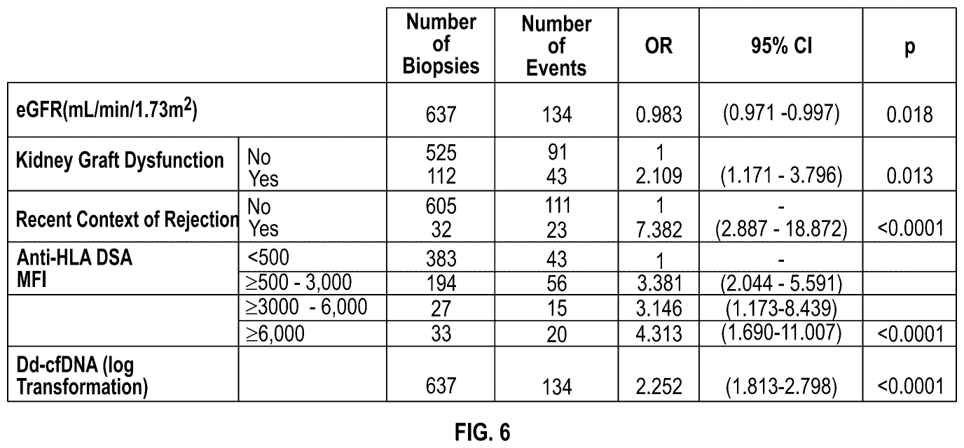

FIG. 6 illustrates a table of example data for a third set of parameters of a cohort dataset selected from a second set of parameters, according to embodiments of the disclosure. Example third set of parameters may comprise parameters, such as (but not limited to), dd-cfDNA levels or concentrations, allograft dysfunction, recent transplant rejection information, and anti-HLA DSA MFI.

In step 410 (of FIG. 4B), the machine-learning model may generate one or more parameter weights corresponding to the third set of parameters of the cohort dataset. For example, the machine-learning model may generate seven parameter weights for seven parameters of the third set of parameters. The parameter weights may be selected and may correspond to the degree of association of the individual parameters to the allograft status (e.g., predicted likelihood of allograft failure). For example, a first parameter may have a corresponding first parameter weight, and a second parameter may have a corresponding second parameter weight. The first parameter may have a higher degree of association to the predicted probability than the second parameter, as reflected by the first parameter weight have a higher value than the second parameter weight. Other relationships and methods for determining parameter weights may also be used. The parameter weights may be calculated using a multivariable logistic regression model.

Embodiments of the disclosure may include training the machine-learning model. The machine-learning model may be trained by using a derivation dataset for a derivation cohort in analyzing the first set of parameters. The machine-learning model may receive the derivation dataset for the derivation cohort and corresponding derivation cohort transplant rejection information. The machine-learning model may also receive a first set of parameters and may analyze the first set of parameters for associations between the derivation dataset and the corresponding (derivation cohort) transplant rejection information. The first set of parameters may be analyzed individually.

During the training stage, the machine-learning model may select a second set of parameters from the first set of parameters, where the second set of parameters meets one or more first criteria. The machine-learning model may then select a third set of parameters from the second set of parameters. The selected third set of parameters may comprise independent variables associated with transplant rejection and meets one or more second criteria. In some embodiments, the selection of parameters for the third set may be based on degree of association. In some embodiments, the parameters selected for the third set may have a higher degree of association than the parameters not selected. The machine-learning model may use the third set of parameters and may generate one or more parameter weights corresponding to the third set of parameters (of the derivation dataset), as discussed above. The machine-learning model may calculate derivation scores based on the third set of parameters of the derivation dataset and the calculated parameter weight(s).

During the validation stage, the machine-learning model may be tested by applying the one or more parameter weights generated from the derivation dataset (during the training stage) to the third set of parameters of the validation dataset. The validation stage is used to validate the trained model and ensure the outputs of the machine-learning model correspond to the data used to train the model. The machine-learning model may calculate a validation score and/or corresponding allograft status (e.g., predicted probability) based on the third set of parameters of the validation dataset and parameter weights. The performance of the machine-learning model may be determined by comparing the derivation scores and the validation scores. In some embodiments, the performance may be based on the discrimination of a score and the area under its receiver operating characteristic (ROC) curve.

Embodiments of the disclosure may include enforcing one or more rules regarding the selection of parameters. The rules may be enforced by way of the criteria for one or more selection steps. For example, the criteria may involve a threshold number of parameters, a threshold degree of association, a threshold number of cohorts, etc.

If the machine-learning model is not trained adequately, the training data (e.g., derivation dataset) may be revised to provide feedback to the model. The output of the machine-learning model between training iterations may be evaluated by a physician or medical expert to determine which data in the training data should be revised. The physician or medical expert can revise certain data in areas of potential improvement, such as which parameter(s) should have a higher degree of association.

### Example Administration of Immunosuppressive Therapy

Immunosuppressive therapy generally refers to the administration of an immunosuppressant or other therapeutic agent that suppresses immune responses to a transplant recipient. Example immunosuppressant agents may include, for example, anticoagulents, antimalarials, heart drugs, non-steroidal anti-inflammatory drugs (NSAIDs), and steroids including, for example, Ace inhibitors, aspirin, azathioprine, B7RP-1-fc, β-blockers, brequinar sodium, campath-1H, celecoxib, chloroquine, corticosteroids, coumadin, cyclophosphamide, cyclosporin A, DHEA, deoxyspergualin, dexamethasone, diclofenac, dolobid, etodolac, everolimus, FK778, feldene, fenoprofen, flurbiprofen, heparin, hydralazine, hydroxychloroquine, CTLA-4 or LFA3 immunoglobulin, ibuprofen, indomethacin, ISAtx-247, ketoprofen, ketorolac, leflunomide, meclophenamate, mefenamic acid, mepacrine, 6-mercaptopurine, meloxicam, methotrexate, mizoribine, mycophenolate mofetil, naproxen, oxaprozin, Plaquenil, NOX-100, prednisone, methyprenisone, rapamycin (sirolimus), sulindac, tacrolimus (FK506), thymoglobulin, tolmetin, tresperimus, UO126, and antibodies including, for example, alpha lymphocyte antibodies, adalimumab, anti-CD3, anti-CD25, anti-CD52 anti-IL2R, and anti-TAC antibodies, basiliximab, daclizumab, etanercept, hu5C8, infliximab, OKT4, and natalizumab.

In some embodiments, no change in predicted probability or variance of the predicted probability over time may indicate no need to adjust immunosuppressive therapy being administered to the transplant recipient, or that the immunosuppressive therapy being administered may be maintained. The decision to maintain immunosuppressive therapy being administered to a transplant recipient may be based on additional clinical factors, such as the health of the transplant recipient. In some embodiments, immunosuppressive therapy being administered to the transplant recipient is maintained.

In some embodiments, adjustment of immunosuppressive therapy includes changing the type or form of immunosuppressant or other immunosuppressive therapy being administered to the transplant recipient. In some embodiments where the transplant recipient is not receiving immunosuppressive therapy, the methods of the present disclosure may indicate a need to begin administering immunosuppressive therapy to the transplant recipient.

Other transplant-related therapies include treatments or therapies besides transplantation or immunosuppressive therapy that are administered to a recipient of a transplant to promote survival of the transplant or to treat transplant-related symptoms (e.g., cytokine release syndrome, neurotoxicity). Examples of other transplant-related therapies include, but are not limited to, administration of antibodies, antigen-targeting ligands, non-immunosuppressive drugs, and other agents that stabilize or destabilize components of transplants that are critical to transplant activity or that directly activate or inhibit one or more transplant activity. These activities may include the ability to induce an immune response, recognize particular antigens, replicate, and/or induce repair of damaged tissues. Adjusting immunosuppressive therapy may be combined with adjusting, initiating, or discontinuing other transplant-related therapies.

The methods of the present disclosure may predict the probability of transplant rejection in a transplant recipient or provide a projection of the risk in a reference set of transplant recipients. The predicted probability and/or projection can be used to inform the need to adjust monitoring of the transplant recipient. In general, changes in predicted risk over time are informative with regard to determining a need to adjust monitoring of a transplant recipient. In some embodiments, determining the status of a transplant, as described above, is informative with regard to determining a need to adjust monitoring of a transplant recipient.

Depending on the status of the transplant, monitoring of the transplant recipient may be adjusted accordingly. For example, monitoring may be adjusted by increasing or decreasing the frequency of monitoring, as appropriate. Monitoring may be adjusted by altering the means of monitoring, for example, by altering the metric that is used to monitor the transplant recipient.

### Example System for Calculating Predicted Probability of Transplant Rejection or Generating a Projection

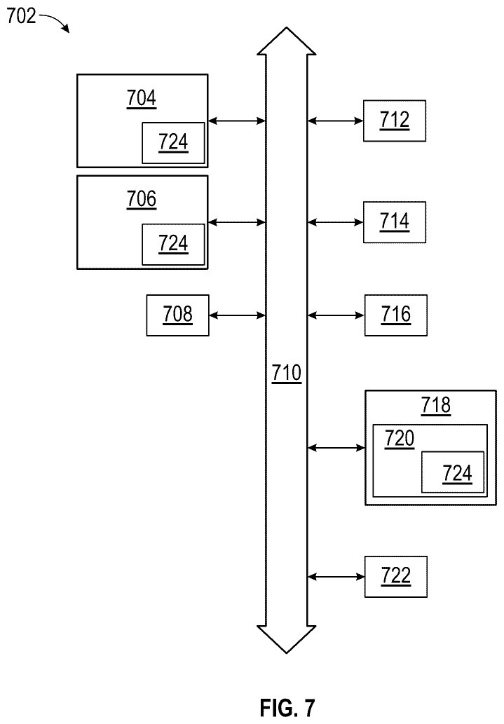

The system and methods discussed above may be implemented by a device. FIG. 7 illustrates an example device that implements the above disclosed system and methods, according to embodiments of the disclosure. The device 702 may be a portable electronic device, such as a cellular phone, a tablet computer, a laptop computer, or a wearable device. The device 702 can include a processor 704 (e.g., a central processing unit (CPU), a graphics processing unit (GPU), or both), a main memory 706 (e.g., read-only memory (ROM), flash memory, dynamic random access memory (DRAM) such as synchronous DRAM (SDRAM) or Rambus DRAM (RDRAM), etc.), and a static memory 708 (e.g., flash memory, static random access memory (SRAM), etc.), which can communicate with each other via a bus 710.

The device 702 may also include a display 712, an input/output device 714 (e.g., a touch screen), a transceiver 716, and storage 718. Storage 718 includes a machine-readable medium 720 on which is stored one or more sets of instructions 724 (e.g., software) embodying any one or more of the methodologies or functions described herein. The software may also reside, completely or at least partially, within the main memory 706 and/or within the processor 704 during execution thereof by the computer 702, the main memory 706, and the processor 704 also constituting machine-readable media. The software may further be transmitted or received over a network via a network interface device 722.

While the machine-readable medium 720 is shown in an embodiment to be a single medium, the term “machine-readable medium” should be taken to include a single medium or multiple media (e.g., a centralized or distributed database and/or associated caches and servers) that store the one or more sets of instructions. The term “machine-readable medium” shall also be taken to include any medium that is capable of storing, encoding, or carrying a set of instructions for execution by the machine and that causes the machine to perform any one or more of the methodologies of the present invention. The term “machine-readable medium” shall accordingly be taken to include, but not be limited to, solid-state memories, optical and magnetic media, and carrier wave signals.

The system and methods described herein and its data can be stored in storage 718, main memory 706, static memory 708, or a combination thereof. The display 712 may be used to present a user interface to a physician or medical expert, and the input/output device 714 may be used to receive input (e.g., clicking on a graphic representative of a microblog) from the physician or medical expert. The transceiver 716 may be configured to communicate with a network, for example.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A computer-implemented method for determining a risk of transplant rejection using a machine-learning system, the method comprising:
receiving, via a computer or an input function, transplant recipient data of a transplant recipient comprising a set of parameters, the set of parameters comprising donor-derived cell-free DNA (dd-cfDNA);
receiving one or more parameter weights;
calculating a score based on the transplant recipient data and the one or more parameter weights; and
calculating a predicted probability of whether or an extent to which transplant rejection in the transplant recipient will occur based on the score.

2. The computer-implemented method of claim 1, wherein the set of parameters of the transplant recipient data further comprises one or more of:
one or more clinical parameters comprising time post-transplantation to evaluation;
one or more functional parameters comprising estimated glomerular filtration rate (eGFR), creatinine, proteinuria, or a combination thereof;
one or more immunological parameters comprising donor-specific antibody mean fluorescence intensity, number of anti-human leukocyte antigens (HLA) mismatches, or both;
one or more transplant recipient characteristics comprising transplant recipient age, donor organ infection information, or both;
one or more transplant characteristics comprising prior transplant information, previous rejection information, or
a combination thereof.

3. The computer-implemented method of claim 1, wherein the set of parameters does not comprise a histological parameter.

4. The computer-implemented method of claim 1, further comprising:
generating a projection of the transplant recipient data in a reference set, wherein the reference set comprises one or more other transplant recipients having one or more common characteristics.

5. The computer-implemented method of claim 4, wherein the generated projections are used to interpret the transplant rejection mechanism, and/or to guide treatment.

6. The computer-implemented method of claim 1, wherein calculating a score comprises:
for each parameter weight, multiplying the parameter weight by a corresponding parameter of the transplant recipient data; and
calculating the score from a summation of the multiplications.

7. The computer-implemented method of claim 1, wherein calculating a predicted probability comprises:
determining an intercept of a multivariable logistic regression model; and
calculating the predicted probability from the score and the intercept.

8. The computer-implemented method of claim 1, wherein the one or more parameters weights are received from a machine-learning model trained to:
acquire a cohort dataset comprising a first set of model parameters and cohort transplant rejection information of transplant recipients in a cohort;
analyze the first set of model parameters for associations between the cohort dataset and the corresponding cohort transplant rejection information;
select one or more subsequent set of model parameters from the first set of model parameters or a preceding set of model parameters;
select a last set of model parameters from the one or more subsequent set of model parameters, wherein the last set of model parameters comprises independent variables associated with transplant rejection and meets one or more second criteria; and
generate the one or more parameter weights corresponding to the last set of model parameters of the cohort dataset.

9. The computer-implemented method of claim 8, wherein the first or the one or more subsequent set of model parameters, or both, comprises dd-cfDNA, and wherein the one or more subsequent set of model parameters meets one or more first criteria.

10. The computer-implemented method of claim 8, wherein the first set of model parameters further comprises one or more of:
one or more clinical parameters comprising graft dysfunction, time since last transplant rejection, time from transplant to evaluation, or a combination thereof;
one or more functional parameters comprising estimated glomerular filtration rate (eGFR), creatinine, proteinuria, or a combination thereof;
one or more immunological parameters comprising donor-specific antibody mean fluorescence intensity, number of anti-human leukocyte antigens (HLA) mismatches, or a combination thereof;
one or more recipient and donor characteristics comprising recipient age, recipient gender, donor organ infection information, or a combination thereof;
one or more transplant characteristics comprising donor age, donor gender, donor type, prior transplant information, cold ischemia time, dual transplant kidney information, or a combination thereof or
a combination thereof.

11. The computer-implemented method of claim 8, wherein the one or more subsequent set of model parameters comprises one or more of:
dd-cfDNA, graft dysfunction, recent transplant rejection information, time post-transplantation to evaluation, estimated glomerular filtration rate (eGFR), proteinuria, donor-specific antibody mean fluorescence intensity, recipient age, recipient gender, donor age, donor gender, donor type, prior transplant information, cold ischemia time, dual transplant information, number of anti-human leukocyte antigens (HLA) mismatches, or a combination thereof.

12. The computer-implemented method of claim 8, wherein the last set of model parameters comprises one or more of: dd-cfDNA, estimated glomerular filtration rate (eGFR), graft dysfunction, recent transplant rejection information, donor-specific antibody mean fluorescence intensity, proteinuria, or a combination thereof.

13. The computer-implemented method of claim 8, wherein analyze the first set of model parameters for associations comprises analyze whether or an extent to which a parameter of the first set of model parameters discriminates between a presence or an absence of transplant rejection in the cohort transplant rejection information.

14. The computer-implemented method of claim 8, wherein the model parameters of the first set are analyzed individually.

15. The computer-implemented method of claim 8, wherein analyze the first set of model parameters for associations comprises determine associations between the dd-cfDNA and one or more of: cause of end stage renal disease, type of transplant rejection, or a combination thereof.

16. The computer-implemented method of claim 8, wherein select the last set of model parameters comprises:
perform backward selection by analyzing whether or an extent to which a parameter of the one or more subsequent set of model parameters discriminates between a presence or an absence of transplant rejection in the cohort transplant rejection information; and
compare the individual analyses to select the last set of model parameters.

17. A system for classifying a status of a transplant comprising:
a scoring unit that:
receives transplant recipient data of a transplant recipient comprising a set of parameters, the set of parameters comprising donor-derived cell-free DNA (dd-cfDNA);
receives one or more parameter weights;
calculates a score based on the transplant recipient data and the one or more parameter weights; and
calculates a predicted probability of whether or an extent to which transplant rejection in the transplant recipient will occur based on the score.

18. The system of claim 17, wherein the set of parameters of the transplant recipient data further comprises one or more of:
one or more clinical parameters comprising time post-transplantation to evaluation;
one or more functional parameters comprising estimated glomerular filtration rate (eGFR), creatinine, proteinuria, or a combination thereof;
one or more immunological parameters comprising donor-specific antibody mean fluorescence intensity, number of anti-human leukocyte antigens (HLA) mismatches, or both;
one or more transplant recipient characteristics comprising transplant recipient age, donor organ infection information, or both;
one or more transplant characteristics comprising prior transplant information, previous rejection information, or both.

19. The system of claim 17, wherein the set of parameters does not comprise a histological parameter.

20. The system of claim 17, further comprising a unit that generates a projection of the transplant recipient data in a reference set, wherein the reference set comprises one or more other transplant recipients having one or more common characteristics.

21. The system of claim 20, wherein the generated projections are used to interpret the transplant rejection mechanism, and/or to guide treatment.

22. The system of claim 17, wherein calculates a score comprises:
for each parameter weight, multiplying the parameter weight by a corresponding parameter of the transplant recipient data; and
calculating the score from a summation of the multiplications.

23. The system of claim 17, wherein calculates a predicted probability comprises:
determining an intercept of a multivariable logistic regression model; and
calculating the predicted probability from the score and the intercept.

24. The system of claim 17, wherein the one or more parameters weights are received from a machine-learning model trained to:
acquire a cohort dataset comprising a first set of model parameters and cohort transplant rejection information of transplant recipients in a cohort;
analyze the first set of model parameters for associations between the cohort dataset and the corresponding cohort transplant rejection information;
select one or more subsequent set of model parameters from the first set of model parameters or a preceding set of model parameters;
select a last set of model parameters from the one or more subsequent set of model parameters, wherein the last set of model parameters comprises independent variables associated with transplant rejection and meets one or more second criteria; and
generate the one or more parameter weights corresponding to the last set of model parameters of the cohort dataset.

25. The system of claim 24, wherein the first or the one or more subsequent set of model parameters, or both, comprises dd-cfDNA, and wherein the one or more subsequent set of model parameters meets one or more first criteria.

26. The system of claim 24, wherein the first set of model parameters further comprises one or more of:
one or more clinical parameters comprising graft dysfunction, time since last transplant rejection, time from transplant to evaluation, or a combination thereof;
one or more functional parameters comprising estimated glomerular filtration rate (eGFR), creatinine, proteinuria, or a combination thereof;
one or more immunological parameters comprising donor-specific antibody mean fluorescence intensity, number of anti-human leukocyte antigens (HLA) mismatches, or a combination thereof;
one or more recipient and donor characteristics comprising recipient age, recipient gender, donor organ infection information, or a combination thereof;
one or more transplant characteristics comprising donor age, donor gender, donor type, prior transplant information, cold ischemia time, dual transplant kidney information, or a combination thereof; or
a combination thereof.

27. The system of claim 24, wherein the one or more subsequent set of model parameters comprises one or more of: dd-cfDNA, graft dysfunction, recent transplant rejection information, time post-transplantation to evaluation, estimated glomerular filtration rate (eGFR), proteinuria, donor-specific antibody mean fluorescence intensity, recipient age, recipient gender, donor age, donor gender, donor type, prior transplant information, cold ischemia time, dual transplant information, number of anti-human leukocyte antigens (HLA) mismatches, or a combination thereof.

28. The system of claim 24, wherein the last set of model parameters comprises one or more of: dd-cfDNA, estimated glomerular filtration rate (eGFR), graft dysfunction, recent transplant rejection information, donor-specific antibody mean fluorescence intensity, proteinuria, or a combination thereof.

29. The system of claim 24, wherein analyze the first set of model parameters for associations comprises analyze whether or an extent to which a parameter of the first set of model parameters discriminates between a presence or an absence of transplant rejection in the cohort transplant rejection information.

30. The system of claim 24, wherein the model parameters of the first set are analyzed individually.

31. The system of claim 24, wherein analyze the first set of model parameters for associations comprises determine associations between the dd-cfDNA and one or more of: cause of end stage renal disease, type of transplant rejection, or a combination thereof.

32. The system of claim 24, wherein select the last set of model parameters comprises:
perform backward selection by analyzing whether or an extent to which a parameter of the one or more subsequent set of model parameters discriminates between a presence or an absence of transplant rejection in the cohort transplant rejection information; and
compare the individual analyses to select the last set of model parameters.

33. A non-transitory computer-readable storage medium for determining a risk of transplant rejection using a machine-learning system, the medium storing one or more programs, the one or more programs comprising instructions, which when executed by one or more processors of an electronic device having display, cause the electronic device to carry out the method of claim 1.
